(19) United States
(12) Patent Application Publication
(10) Pub. No.: US 2024/0238645 A1
(43) Pub. Date: Jul. 18, 2024

(54) METHOD FOR DETECTING THE DISTANCE OF PORTIONS OF THE BODY OF A USER FROM A PART OF A GYMNASTIC MACHINE AND GYMNASTIC MACHINE CARRYING OUT THE METHOD

(71) Applicant: Technogym S.p.A, Cesena FC (IT)

(72) Inventor: Enrico VANNONI, Cesena FC (IT)

(73) Assignee: Technogym S.p.A, Cesena FC (IT)

(21) Appl. No.: 18/218,181

(22) Filed: Jul. 5, 2023

(30) Foreign Application Priority Data
Jul. 5, 2022 (IT) 102022000014224

(51) Int. Cl. A63B 24/00; A63B 71/06

(52) U.S. Cl. CPC A63B 24/0062; A63B 71/0619; A63B 2024/0093; A63B 2220/05; A63B 2220/805

(57) ABSTRACT

The present invention relates to a method for detecting the distance of portions of the body of a user (U) from a front part (121) of a gymnastic machine (1) provided with a detecting device (123), arranged on said front part (121) and a logic unit control (C), operatively connected to said detecting device (123), during the execution of a gymnastic exercise on an exercise surface (111), said method comprising the following step:
detecting (100), by means of said detecting device (123), a plurality of distance values of at least one portion of the body (O) of said user (U) from said front part (121) of said gymnastic machine (1);
processing (101), by means of said logic control unit (C), at least one first group of said plurality of distance values of at least one portion of the body (O) of said user (U), for calculating the distance of at least one portion of the chest (O₁) of the user (U) from said detecting device (123);
and/or processing (102), by means of said logic control unit (C), at least one second group of said plurality of distance values of at least one portion of the body (O) of said user (U), for calculating the distance of at least one portion of the right leg (O) of the user (U) from said detecting device (123);
and/or processing (103), by means of said logic control unit (C), at least one third group of said plurality of distance values of at least one portion of the body (O) of said user (U), for calculating the distance of at least one portion of the left leg (O) of the user (U) from said detecting device (123).

The present invention also relates to an exercise machine that implements the method.

### RELATED APPLICATION

This application claims the benefit of priority of Italy Patent Application No. 102022000014224 filed on Jul. 5, 2022, the contents of which are incorporated by reference as if fully set forth herein in their entirety.

### FIELD AND BACKGROUND OF THE INVENTION

The present invention relates to a method of detecting the distance of portion of the body of a user from a part of a gymnastic machine, in particular of a treadmill.

The present invention also relates to the gymnastic machine which implements the method.

More in detail, the invention relates to a method of the aforementioned type, designed and implemented in particular for detecting the distance of several portions of the body of a user, with respect to an upper supporting structure of the treadmill, but which can be used for any gymnastic machine, in which it is necessary to detect the distance of portions of the body of a user.

In the following, the description will be directed to a method implemented in a treadmill machine, during the execution of a walk or a run by a user, but it is clear that the same should not be considered limited to this specific use.

As is well known, gymnastic machines with rotating belts are currently known, comprising an exercise surface, which can be a belt or flat carpet, or which can comprise a plurality of strips so as to form a flat or curved “rolling shutter”, in which measurements of a user's running progress are made in order to determine the quality of the training.

It is in fact of great importance to be able to determine the quality of the training of the user, so that it is on one hand performing, and on the other hand also reliable and safe, for the well-being and health of the user.

Methods for determining the training quality, which requires very complicated and sophisticated detection devices that can cause errors in data collection and processing, are currently known.

In light of the above, it is, therefore, a scope of the present invention to provide a method that allows to at least partially overcome the drawbacks reported with reference to the prior art, and which therefore allows reading values relating to several portions of the body of the user by means of structurally simple devices, which allow the quality of the training to be determined in a precise and reliable way.

A further scope of the present invention is to provide a gymnastic machine required for carrying out the method and the apparatuses that carries out this method.

### SUMMARY OF THE INVENTION

It is, therefore, specific object of the present invention a method for detecting the distance of portions of the body of a user from a front part of a gymnastic machine provided with a detecting device, arranged on said front part and a logic control unit, operatively connected to said detecting device, during the execution of a gymnastic exercise on an exercise surface, said method comprising the following step:
detecting, by means of said detecting device, a plurality of distance values of at least one portion of the body of said user from said front part of said gymnastic machine;
processing, by means of said logic control unit, at least one first group of said plurality of distance values of at least one portion of the body of said user, for calculating the distance of at least one portion of the chest of the user from said detecting device;
and/or processing, by means of said logic control unit, at least one second group of said plurality of distance values of at least one portion of the body of said user, for calculating the distance of at least one portion of the right leg of the user from said detecting device;
and/or processing, by means of said logic control unit, at least one third group of said plurality of distance values of at least one portion of the body of said user, for calculating the distance of at least one portion of the left leg of the user from said detecting device.

Further according to the invention, said processing step comprises the following sub-steps:
selecting, by means of said logic control unit, said first group of said plurality of distance values of at least one portion of the body;
calculating, by means of said logic control unit, said distance of at least one portion of the chest of the user from said detecting device as a statistical data of said first group of values of said plurality of distance values of at least one portion of the body.

Still according to the invention, said method comprises the following step:
comparing, by means of said logic control unit, said current distance data of at least one portion of the chest, calculated in said sub-step of calculating, with a predetermined reference value, corresponding to an optimal distance of the user from said detecting device, on said exercise surface.

Preferably according to the invention, said step of comparing comprises the following sub-steps:
if said current distance data of at least one portion of the chest is higher than said predetermined reference value plus a predetermined threshold value, then commanding the decrease, by means of said logic control unit, of the advancement speed of said exercise surface,
if said current distance data of at least one portion of the chest is included in a range having as lower bound said predetermined reference value minus said predetermined threshold value and as upper bound said predetermined reference value plus said predetermined threshold value, then leave unchanged the advancement speed of said exercise surface;
if said current distance data of at least one portion of the chest is lower than said predetermined reference value minus a predetermined threshold value, then command the increase, by means of said logic control unit, of the advancement speed of said exercise surface.

Always according to the invention, said processing step comprises the following sub-steps:
selecting, by means of said logic control unit, said second group of values of said plurality of distance values of at least one portion of the body;
calculating, by means of said logic control unit, said distance of at least one portion of the right leg of the user from said detecting device as a statistical data of said second group of values of said plurality of distance values of at least one portion of the body.

Further according to the invention, said processing step comprises the following sub-steps:
selecting, by means of said logic control unit, said third group of values of said plurality of distance values of at least one portion of the body;
calculating, by means of said logic control unit, said distance of at least one portion of the left leg of the user from said detecting device as a statistical data of said third group of values of said plurality of distance values of at least one portion of the body.

Still according to the invention, said method comprises the following step:
providing a display of the parameters of the execution of the gymnastic exercise, based on the distance data calculated in said sub-steps of calculating.

Preferably according to the invention, said step of providing a display comprises the following sub-steps:
obtaining the oscillation period of the right leg, from said distance data of at least one portion of the right leg;
obtaining the oscillation period of the left leg, from said distance data of at least one portion of the right leg;
comparing said oscillation period of the right leg and said oscillation period of the left leg;
displaying the result of said comparison by means of a display of said gymnastic machine.

Still according to the invention, said step of providing a display comprises the following sub-steps:
obtaining the amplitude of the oscillation of the left leg, from said distance data of at least one portion of the left leg;
obtaining the amplitude of the oscillation of the right leg, from said distance data of at least one portion of the right leg;
comparing said amplitude of the oscillation of the right leg and said amplitude of the oscillation of the left leg;
displaying said amplitude of the oscillation of the right leg and said amplitude of the oscillation of the left leg or the result of said comparison by means of a display of said gymnastic machine.

Further according to the invention, said step of providing a display comprises the following sub-steps:
obtaining the oscillation period of the right leg, from said distance data of at least one portion of the right leg;
obtaining the oscillation period of the left leg, from said distance data of at least one portion of the left leg;
calculating the running cadence as the inverse of the time that passes between two successive peaks of the oscillation of the right leg and of the oscillation of the left leg;
displaying said cadence by means of a display of said gymnastic machine.

Still according to the invention, said method comprises the following steps:
detecting a plurality of ambient brightness values;
processing said plurality of detected ambient brightness values for calculating an ambient brightness index;
comparing, said ambient brightness index, with a predetermined threshold value;
if said ambient brightness index is greater than said predetermined threshold value, carrying out the steps of the method;
if said ambient brightness index is lower than said predetermined threshold value, activating a safety operating state for said gymnastic machine.

Preferably according to the invention, in said step of detecting, said plurality of distance values of at least one portion of the body of said user is detected in the form of a matrix, in which at least one distance value is associated with each cell. Still according to the invention, said first group of said plurality of distance values of at least one portion of the body of said user is contained in an upper portion of said matrix.

Further according to the invention, said second group of said plurality of distance values of at least one portion of the body of said user is contained in a first lower portion of said matrix.

Still according to the invention, said third group of said plurality of distance values of at least one portion of the body of said user is contained in the cells of a second lower portion of said matrix.

A further object of the present invention is a computer program comprising instructions that, when said program is executed by a computer, cause the execution by said processor of the steps of said method.

A further object of the present invention is a computer-readable storage medium, comprising instructions that, when said program is executed by a computer, cause the execution by said processor of the steps of said method.

A further object of the present invention is a gymnastic machine comprising an exercise surface for the training of a user on said gymnastic machine, the exercise surface having a development direction and an advancement direction parallel to the development direction of the exercise surface, a logic control unit of the movement of the exercise surface, a detecting device, operatively connected to said logic control unit, configured to detect at least one distance value of a portion of the user from said detecting device itself, during the execution of the gymnastic exercise by the user on the exercise surface, said logic control unit being configured to carry out the method for detecting the distance of portions of the body of a user from a part of a gymnastic machine, according to the previous steps of the method.

### BRIEF DESCRIPTION OF THE SEVERAL VIEWS OF THE DRAWINGS

The present invention will be now described, for illustrative but not limitative purposes, according to its preferred embodiments, with particular reference to the figures of the enclosed drawings, wherein:

FIG. 1 shows a side view of a first embodiment of the gymnastic machine that implements the method, object of the present invention;

FIG. 2 shows a top view of the gymnastic machine of FIG. 1;

FIG. 3 shows a perspective view of a second embodiment of the gymnastic machine that implements the method, object of the present invention;

FIG. 4 shows a top view of the gymnastic machine in FIG. 3;

FIG. 5 shows a detecting matrix of a component of the gymnastic machine;

FIG. 6 shows a block diagram of the method of detecting the distance of portions of the body of a user from a part of a gymnastic machine, object of the present invention; and

FIG. 7 shows the trend of signals detected in the gymnastic machine during the execution of the method.

### DESCRIPTION OF SPECIFIC EMBODIMENTS OF THE INVENTION

In the various figures, similar parts will be indicated with the same reference numbers.

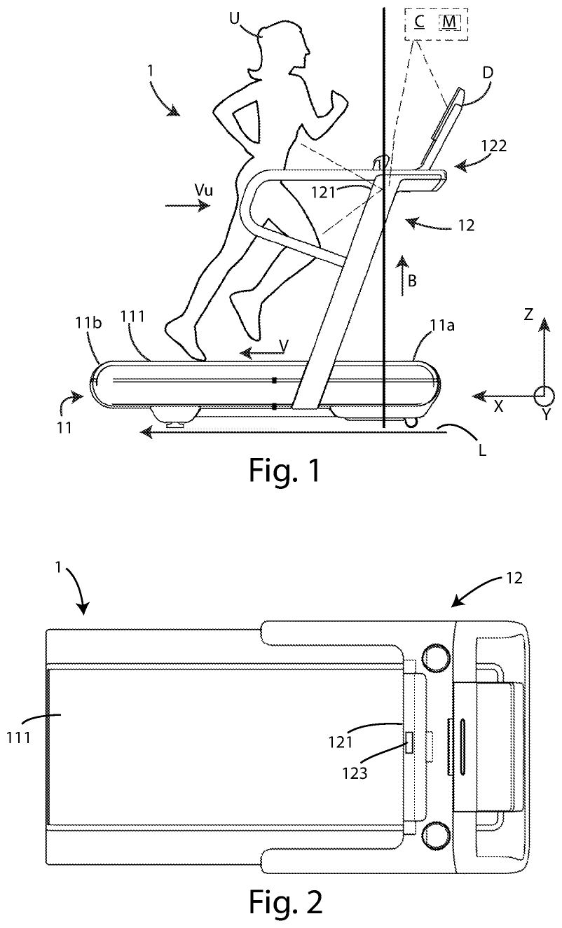

With reference to FIG. 1, the gymnastic machine 1, which implements the method object of the present invention, is in particular a treadmill.

Said treadmill 1 has a supporting base 11 which extends along a longitudinal axis L, parallel to a first Cartesian axis X of a Cartesian reference system XYZ shown in the figure.

Said supporting base 11 has a first end 11ₐ and a second end 11_{b}.

In the first end 11ₐ first rotating element is arranged, not shown in the figure, capable of rotating about a first axis of rotation parallel to a second Cartesian axis Y of the Cartesian reference system.

In the second end 11_{b} second rotating element is arranged, not shown in the figure, capable of rotating about a second axis of rotation parallel to said second Cartesian axis Y of the Cartesian reference system.

Said treadmill 1 comprises an exercise surface 111 for the training of the user U, such as running or walking, or any other exercise that can be performed on said exercise surface 111.

In a first embodiment, said exercise surface 111 is a carpet or a belt.

Said exercise surface 111 is operatively connected to said first and second rotating element, so that, when said first and second rotating elements rotate about the respective first and second axes, said exercise surface 111 is capable of sliding according to an advancement direction V, parallel to said first Cartesian axis X.

Said advancement direction V is opposite to the moving direction Vᵤ of the user U, during the execution of the gymnastic exercise.

In said embodiment, said treadmill comprises a motor, not shown in the figure, capable of rotating said first or said second rotating element, or both, so as to cause the sliding of said exercise surface 111.

Furthermore, said treadmill 1 comprises a logic control unit C, arranged on said treadmill 1, or remote.

Said logic control unit C is capable of controlling the movement of said exercise surface 111.

In particular, said logic control unit C is capable of controlling the operation of said motor.

Furthermore, said logic control unit C comprises a memory unit M, in which parameters and operating data for the operation of the treadmill 1 are stored.

Said treadmill 1 comprises a supporting frame 12, arranged at said first end 11a of said supporting base 11.

Said supporting frame 12 extends according to an axis B substantially orthogonal to the exercise surface 111 of said supporting base 11, and parallel to a third Cartesian axis Z of the Cartesian reference system.

Said supporting frame 12 comprises one or more uprights and tubular elements connected to each other, on which the user U can rest his upper limbs during training or in the rest step.

In particular, said supporting frame 12 comprises a front part 121 that faces the user U, which can be equipped with housings for supporting objects of various types.

Said supporting frame 12 further comprises a user interface 122, operatively connected to said logic control unit C.

Said user interface 122 is configured to allow the user to issue commands to said treadmill 1.

Said user interface 122 can comprise a touchscreen display D or a display D connected to a keyboard or other data entry devices for user U, not shown in the figure.

The display D allows the user U to view both specific contents of the use of the treadmill 1, including contents related to the method of detecting the distance of the portions of the body that will be described below, and multimedia contents of entertainment or services for user U.

With reference now to FIG. 2, said treadmill 1 comprises a detecting device 123 for the distance of portions of the body of the user U.

Said detection device 123 is operatively connected to said logic control unit C.

Said detecting device 123 is arranged on said supporting frame 12.

In particular, in the preferred embodiment that is being described, said detecting device 123 is arranged in said front part 121.

However, without departing from the scope of protection of the present invention, said detecting device 123 can also be arranged in different positions, such as for example in the lateral or rear part of said supporting frame 12 of said treadmill 1.

Said detecting device 123 is a distance sensor, configured to detect a distance value O of a portion of the user U from himself, during the training on the treadmill 1.

Portion of the body of the user U here means at least a portion of the chest, and/or right leg, and/or left leg of the user U. In particular, the operation of said distance sensor 123 is based on the time-of-flight principle, or the measurement of the distance O between the sensor and the user U is based on the difference of time between the emission of a signal and its return to the sensor, after being reflected by the user U.

The signal can be of electromagnetic or acoustic nature, especially light or sound.

In the embodiment described, the sensor used is of the optical type, therefore the signal used is of an electromagnetic nature.

The distance sensor 123 detects values and outputs a packet of values comprising several distance values O and/or any further values.

In particular, in this embodiment, the distance sensor 123 outputs a packet of values in the form of a matrix M.

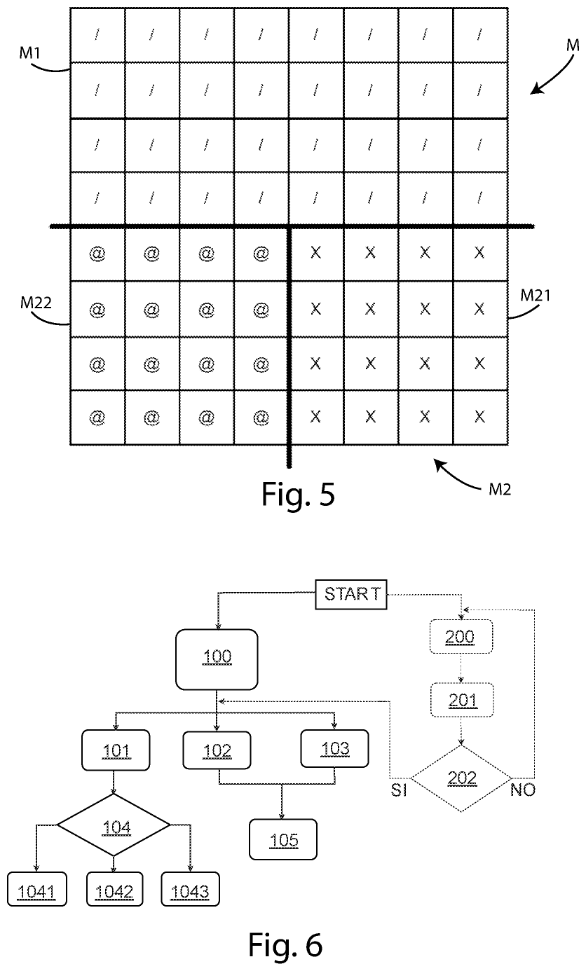

With reference to FIG. 5, in particular, said matrix M has a size of 8×8 cells and will be described in detail below.

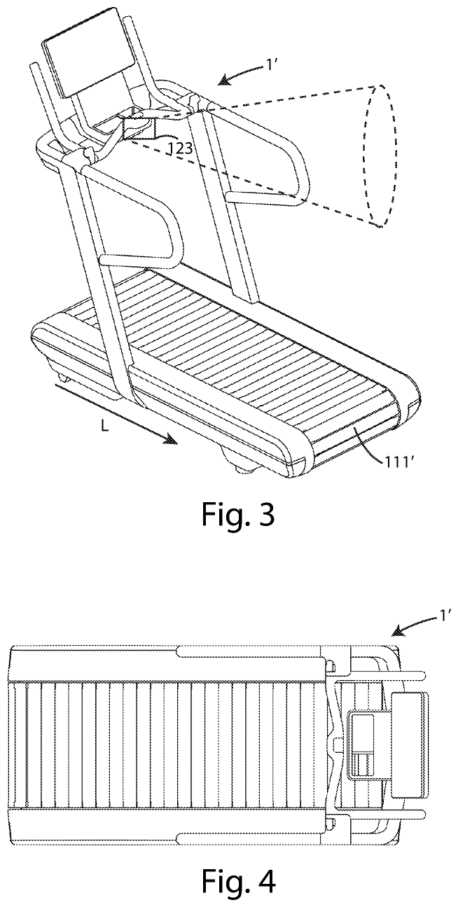

With reference to FIGS. 3 and 4, in a second embodiment, said gymnastic machine 1′ varies if compared to the first embodiment described above, only for the exercise surface 111′.

In the second embodiment, said exercise surface 111′ comprises a plurality of strips arranged transversely to the longitudinal axis L, giving the exercise surface 111′ a so-called “roller shutter” shape.

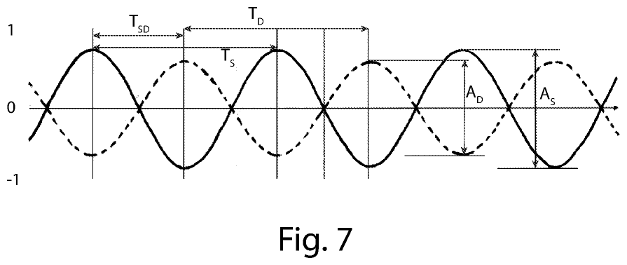

With reference to FIG. 6, the method of detecting the distance O of portions of the body of a user U from a part of a gymnastic machine 1, object of the present invention, provides the execution of the following steps, by means of said logic control unit U.

The method implements two different functions, the first is the calculation of the distance of at least a portion of the chest O₁ of the user U from the distance sensor 123, and the second is the calculation of the distance of a portion of the right leg O₂ and of the left leg O₃ of the user U, from the distance sensor 123. ₂₃

The method is performed by said logic control unit C, with the values sent by said distance sensor 123 to said logic control unit C.

In particular, as previously described, the portions of the body of the user U are at least a portion of the chest, right leg, and left leg.

Furthermore, by part of the gymnastic machine 1 we mean the supporting frame 12, in particular the front part 121.

### First Functionality

The first functionality involves the execution of the following steps.

In a detecting step 100, said distance sensor 123 detects the distance O of said user U in the form of a matrix M.

In particular said matrix M has a dimension of 8×8 pixels.

In particular, each cell of the matrix M is associated with a distance value O measured by a portion of the body of the user U.

Each cell of the matrix M can also be associated with an ambient brightness index I_{L}.

The sampling frequency of the distance sensor 123 can vary from 5 to 20 times per second, and, for each detection, the values are collected and the matrix M is generated.

In a first processing step 101 the values of said matrix M are processed.

In particular, in a first sub-step 1011 the distance of a portion of the chest O₁ of the user U is calculated from the distance sensor 123, by selecting a first group of detected distance values.

In particular, an upper portion M1 of the matrix M, which contains said first group of detected distance values, is selected.

In one embodiment, the dimensions of said upper portion M1 are 4×8 pixels or cells.

Without departing from the scope of protection of the present invention, the dimensions of said upper portion M1 may have different values.

For example, the dimensions of said upper portion M1 can be 5×8 or 6×8.

As shown in FIG. 5, each cell of the upper portion M1 contains the distance values of a portion of the chest O₁ of the user, indicated with the symbol “/”.

In a second sub-step 1012 a statistical datum of the distance of a portion of the chest O₁ measured from each cell of the upper portion M₁ of the matrix M for each sampling cycle, i.e. for each time instant, is calculated.

Said statistical datum can be, for example, the mode, median, or mean, which represents the statistical distribution of the first group of distance values in the upper portion M₁ of the matrix M.

In a comparing step 104, the current distance value of a portion of the chest O₁ is compared with a reference value O_{1R}, which corresponds to an optimal positioning distance of the user U on said exercise surface 111. If the current distance value of a portion of the chest O₁ s higher than the ₁ reference value O_{1R} plus a predetermined threshold value S, i.e., if O₁>O_{1R}+S, then in a first controlling step 1041, said logic control unit C is configured to determine the decrease of the advancement speed of the exercise surface 111.

If the current distance value of a portion of the chest O₁ is within a range determined by O_{1R}−S<O₁<O_{1R}+S, then in a second controlling step 1042, said logic control unit C is configured to leave unchanged the advancement speed of the exercise surface 111.

If the current distance value of a portion of the chest O₁ is lower than the reference value O_{1R} minus a predetermined threshold value S, i.e. if O₁<O_{1R}−S, then in a third controlling step 1043, said logic control unit C is configured to determine the increase of the advancement speed of the exercise surface 111.

### Second Functionality

The second functionality involves the execution of the following steps.

Still referring to FIGS. 5 and 6, after said detecting step 100, the values of said matrix M are processed, in a second processing step 102, selecting a second group of detected distance values.

In particular, in a first sub-step 1021, the distance of a portion of the right leg O₂ of the user U from distance sensor 123 is calculated.

In order to determine a value of the distance of a portion of the right leg O₂, a first lower portion M₂₁ of said matrix M, which contains said second group of detected distance values, is selected.

In particular, the dimensions of said first lower portion M₂₁ are 4×4 pixels.

Without departing from the scope of protection of the present invention, the dimensions of said first lower portion M₂₁ may have different values.

For example, the dimensions of said first lower portion M₂₁ can be 4×5 or 4>3.

As shown in FIG. 5, each cell of the first lower portion M21 contains the distance values of a portion of the right leg O₂ of the user, indicated with the symbol “X”.

In a second sub-step 1022, it is calculated a statistical datum of the distance of a portion of the right leg O₂ measured from each cell of the first upper lower portion M₂₁ of the matrix M for each sampling cycle, i.e., for each time instant.

Said statistical datum can be, for example, the mode, median or mean, which represent the statistical distribution of the first group of distance values in the upper portion M₁ of the matrix M.

Still referring to FIGS. 5 and 6, after said detecting step 100, in a third processing step 103, the values of said matrix M are processed, selecting a third group of detected distance values.

In particular, in a first sub-step 1031, it is calculated the distance of a portion of the left leg O₃ of the user U from the distance sensor 123.

A second lower portion M22 of said matrix M is selected, in order to determine a value of the distance of a portion of the left leg O₃.

In particular, the dimensions of said second lower portion M22 are 4×4 pixels.

Without departing from the scope of protection of the present invention, the dimensions of said second lower portion M22 may have different values.

For example, the dimensions of said second lower portion M22 can be 4×5 or 4×3.

As shown in FIG. 5, each cell of the second lower portion M22 contains the third group of distance values of a portion of the left leg O₃ of the user, indicated with the symbol “@”.

In a second sub-step 1032, a statistical datum of the distance of a portion of the left leg O₃, measured by each cell of the second lower portion M22 of the matrix M for each sampling cycle, is calculated, i.e. for each time instant.

Said statistical datum can be, for example, the mode, median or mean, which represent the statistical distribution of the third group of distance values in the second lower portion M22.

In an evaluating step 105, said distances of a portion of the right leg O₂ and a portion of the left leg O₃ are processed, in order to provide the user information on the characteristics and quality of the execution of the exercise in real-time, by means of said display D.

Alternatively, with reference to FIG. 7, memorizing, in said memory unit M of said logic control unit U, the trend of the distance values of a portion of the right leg O₂, indicated in the figure with a dashed line trace, and the distance values of a portion of the left leg O₃, indicated in the figure with a continuous line, cyclic and out-of-phase curves are obtained, from which it is possible to obtain characteristic parameters.

In particular, the characteristic parameters are: the oscillation amplitude of the right leg, indicated with A_{D}, the oscillation amplitude of the left leg, indicated with A_{S}, the oscillation period of the right leg, indicated with T_{D} and the oscillation period of the left leg, indicated with T_{S}.

By calculating the distance between two succeeding peaks T_{SD} of the right leg oscillation period T_{D} and the left leg oscillation period T_{S}, the value of the cadence C=1/T_{SD}, which indicates the number of steps taken in one minute, is obtained.

The cadence C can be compared with recommended reference values based on the height or other anthropometric parameters of the user U, in order to provide the user with suggestions on how to improve the quality of the run, by means of this display D.

Comparing the periods T_{D} e T_{S} and the oscillations A_{D} e A_{S} with pre-established experimental data, indications for the user U on the quality of training can be displayed, such as the symmetry of use of the lower limbs during running or the detection of asymmetry of these.

Examples of visualizations can be arrows or indicators that indicate the deviation of the calculated values from a symmetry condition.

For example, the greater the oscillation period T_{D} compared to the oscillation period T_{S} or vice versa, the less symmetrical the run of the user will be.

In an optional step 200, a brightness control step of the values detected by the distance sensor 123 can also be performed.

A valuation of the ambient brightness index I_{L} is carried out which is the number of photons received by the pixels of the distance sensor 123.

As previously described, the cells of said matrix M, in addition to detecting distance values, also memorize ambient brightness values I_{L}.

In a processing step 201, said ambient brightness values I_{L} detected by said matrix M are processed.

In a verification step 202, the ambient brightness values I_{L} processed in said processing step 201 are compared with a predetermined threshold value.

Said pre-established threshold value relates to a determined distance sensor 123 used.

If the ambient brightness index I_{L} is greater than said pre-established threshold value, then some optional automatic functions, which can be performed by said treadmill 1, such as the control of the rotation speed of said exercise surface 111, are enabled.

If the ambient brightness index I_{L} is lower than said pre-established threshold value, said treadmill 1 enters a “safety” operating state, which provides for disabling some accessory automatic functions that can be performed by said treadmill 1 itself.

In particular, if the ambient brightness data I_{L} processed are higher than said pre-established threshold value, then it is proceed with the execution of the steps of the method starting from said detecting step 100.

If the ambient brightness data I_{L} processed are lower than said pre-established threshold value, then the steps of the method starting from said detecting step 100 are disabled.

The operation of the treadmill 1 described above is as follows.

When a user U intends to perform a workout, s/he accesses said treadmill 1 exercise surface 111.

By means of the display D s/he actuates the operation, and the motor rotates said first rotating element about its rotation axis.

The rotation of the first rotating element causes the rotation of said exercise surface 111, which also rotates the second rotating element around its rotation axis.

The user U can then perform the walking or running gymnastic exercise.

Said distance sensor 123 detects said distance values O of portions of the user U and sends said processed values to said logic control unit U, which follows the steps of the method described above.

### Advantages

As is apparent from the above description, the method object of the present invention allows detecting and determining the distance of different portions of the body of a user from a part of a gymnastic machine, during the performance of a gymnastic exercise.

Furthermore, following the determination of said distances, the method allows actuating some controls on said machine.

The present invention has been described for illustrative but not limitative purposes, according to its preferred embodiments, but it is to be understood that modifications and/or changes can be introduced by those skilled in the art without departing from the relevant scope as defined in the enclosed claims.

1. Method for detecting the distance of portions of the body of a user (U) from a front part (121) of a gymnastic machine (1) provided with a detecting device (123), arranged on said front part (121) and a logic unit control (C), operatively connected to said detecting device (123), during the execution of a gymnastic exercise on an exercise surface (111), said method comprising the following step:
detecting (100), by means of said detecting device (123), a plurality of distance values of at least one portion of the body (O) of said user (U) from said front part (121) of said gymnastic machine (1);
said method being characterized in that it comprises the following further steps:
processing (101), by means of said logic control unit (C), at least one first group of said plurality of distance values of at least one portion of the body (O) of said user (U), for calculating the distance of at least one portion of the chest (O₁) of ₁ the user (U) from said detecting device (123);
and/or processing (102), by means of said logic control unit (C), at least one second group of said plurality of distance values of at least one portion of the body (O) of said user (U), for calculating the distance of at least one portion of the right leg (O) of the user (U) from said detecting device (123);
and/or processing (103), by means of said logic control unit (C), at least one third group of said plurality of distance values of at least one portion of the body (O) of said user (U), for calculating the distance of at least one portion of the left leg (O) of the user (U) from said detecting device (123).

2. Method according to claim 1, wherein said processing step (101) comprises the following sub-steps:
selecting (1011), by means of said logic control unit (C), said first group of said plurality of distance values of at least one portion of the body (O);
calculating (1012), by means of said logic control unit (C), said distance of at least one portion of the chest (O₁) of the user (U) from said detecting device (123) as a statistical data of said first group of values of said plurality of distance values of at least one portion of the body (O).

3. Method according to claim 2, wherein it comprises the following step:
comparing (104), by means of said logic control unit (C), said current distance data of at least one portion of the chest (O1), calculated in said sub-step of calculating (1012), with a predetermined reference value (O_{1R}), corresponding to an optimal distance of the user (U) from said detecting device (123), on said exercise surface (111).

4. Method according to claim 3, wherein said step of comparing (104) comprises the following sub-steps:
if said current distance data of at least one portion of the chest (O₁) is higher than said predetermined reference value (O_{1R}) plus a predetermined threshold value (S), then commanding the decrease (1041), by means of said logic control unit (C), of the advancement speed of said exercise surface (111).
if said current distance data of at least one portion of the chest (O₁) is included in a range having as lower bound said predetermined reference value (O_{1R}) minus said predetermined threshold value (S) and as upper bound said predetermined reference value (O_{1R}) plus said predetermined threshold value (S), then leave unchanged (1042) the advancement speed of said exercise surface (111);
if said current distance data of at least one portion of the chest (O₁) is lower than said predetermined reference value (O_{1R}) minus a predetermined threshold value (S), then command the increase (1043), by means of said logic control unit (C), of the advancement speed of said exercise surface (111).

5. Method according to claim 1, wherein said processing step (102) comprises the following sub-steps:
selecting (1021), by means of said logic control unit (C), said second group of values of said plurality of distance values of at least one portion of the body (O);
calculating (1022), by means of said logic control unit (C), said distance of at least one portion of the right leg (O₂) of the user (U) from said detecting device (123) as a statistical data of said second group of values of said plurality of distance values of at least one portion of the body (O).

6. Method according to claim 1, wherein said processing step (103) comprises the following sub-steps:
selecting (1031), by means of said logic control unit (C), said third group of values of said plurality of distance values of at least one portion of the body (O);
calculating (1032), by means of said logic control unit (C), said distance of at least one portion of the left leg (O₃) of the user (U) from said detecting device (123) as a statistical data of said third group of values of said plurality of distance values of at least one portion of the body (O).

7. Method according to claim 5, wherein it comprises the following step:
providing a display (105) of the parameters of the execution of the gymnastic exercise, based on the distance data calculated in said sub-steps of calculating (1022; 1032).

8. Method according to claim 7, wherein said step of providing a display (105) comprises the following sub-steps:
obtaining the oscillation period (T_{D}) of the right leg, from said distance data of at least one portion of the right leg (O₂);
obtaining the oscillation period (T_{S}) of the left leg, from said distance data of at least one portion of the right leg (O₂);
comparing said oscillation period (T_{D}) of the right leg and said oscillation period (T_{S}) of the left leg;
displaying the result of said comparison by means of a display (D) of said gymnastic machine (1).

9. Method according to claim 7, wherein said step of providing a display (105) comprises the following sub-steps:
obtaining the amplitude of the oscillation (A_{S}) of the left leg, from said distance data of at least one portion of the left leg (O₃);
obtaining the amplitude of the oscillation (A_{D}) of the right leg, from said distance data of at least one portion of the right leg (O₂);
comparing said amplitude of the oscillation (A_{D}) of the right leg and said amplitude of the oscillation (A_{S}) of the left leg;
displaying said amplitude of the oscillation (A_{D}) of the right leg and said amplitude of the oscillation (A_{S}) of the left leg or the result of said comparison by means of a display (D) of said gymnastic machine (1).

10. Method according to claim 7, wherein said step of providing a display (105) comprises the following sub-steps:
obtaining the oscillation period (T_{D}) of the right leg, from said distance data of at least one portion of the right leg (O₂);
obtaining the oscillation period (T_{S}) of the left leg, from said distance data of at least one portion of the left leg (O₃);
calculating the running cadence (C) as the inverse of the time (T_{SD}) that passes between two successive peaks of the oscillation (T_{D}) of the right leg and of the oscillation (T_{S}) of the left leg;
displaying said cadence (C) by means of a display (D) of said gymnastic machine (1).

11. Method according to claim 1, wherein it comprises the following steps:
detecting (200) a plurality of ambient brightness values;
processing (201) said plurality of detected ambient brightness values for calculating an ambient brightness index (I_{L});
comparing (202), said ambient brightness index (I_{L}), with a predetermined threshold value;
if said ambient brightness index (I_{L}) is greater than said predetermined threshold value, carrying out the steps of the method according to any one of claims 1-10;
if said ambient brightness index (I_{L}) is lower than said predetermined threshold value, activating a safety operating state for said gymnastic machine (1).

12. Method according to claim 1, wherein in said step of detecting (100), said plurality of distance values of at least one portion of the body (O) of said user (U) is detected in the form of a matrix (M), in which at least one distance value (O) is associated with each cell.

13. Method according to claim 12, wherein said first group of said plurality of distance values of at least one portion of the body (O) of said user (U) is contained in an upper portion (M1) of said matrix (M).

14. Method according to claim 12, wherein said second group of said plurality of distance values of at least one portion of the body (O) of said user (U) is contained in a first lower portion (M21) of said matrix (M).

15. Method according to claim 12, wherein said third group of said plurality of distance values of at least one portion of the body (O) of said user (U) is contained in the cells of a second lower portion (M22) of said matrix (M).

16. Computer program comprising instructions that, when said program is executed by a computer, cause the execution by said processor of the steps of said method according to claim 1.

17. Computer readable storage medium, comprising instructions that, when said program is executed by a computer, cause the execution by said processor of the steps of said method according to claim 1.

18. Gymnastic machine (1) comprising:
an exercise surface (111) for the training of a user (U) on said gymnastic machine (1), the exercise surface (111) having a development direction (L) and an advancement direction (V) parallel to the development direction (L) of the exercise surface (111);
a logic control unit (C) of the movement of the exercise surface (111);
a detecting device (123), operatively connected to said logic control unit (C), configured to detect at least one distance value (O) of a portion of the user (U) from said detecting device (123) itself, during the execution of the gymnastic exercise by the user (U) on the exercise surface (111);
said logic control unit (C) being configured to carry out the method for detecting the distance of portions of the body of a user (U) from a part of a gymnastic machine (1) according to claim 1.
